(19) United States
(12) Patent Application Publication
(10) Pub. No.: US 2025/0018072 A1
(43) Pub. Date: Jan. 16, 2025

(54) SYSTEM AND METHOD FOR UV BASED MICROORGANISM INACTIVATION

(71) Applicant: UVI INDOOR LTD., Tel Aviv (IL)

(72) Inventors: Amir HADA, Tel Aviv (IL); Israel AMAR, Tel Aviv (IL)

(21) Appl. No.: 18/712,004

(22) PCT Filed: Nov. 21, 2022
(86) PCT No.: PCT/IL2022/051239

§ 371 Date: May 21, 2024

Related U.S. Application Data
(60) Provisional application No. 63/281,693, filed on Nov. 21, 2021.

(51) Int. Cl. A61L 2/10; A61L 2/24; A61L 2/28

(52) U.S. Cl. CPC A61L 2/10; A61L 2/24; A61L 2/28; A61L 2202/14

(57) ABSTRACT

Disclosed herein is a UV based microorganism inactivation system, comprising: at least one UV source, configured to emit UV radiation; at least one UV sensor, located at a known location with respect to the at least one UV source; and a controller. The controller is configured to: receive UV radiation measurements from the at least one UV sensor; receive information related to an internal space accommodating the at least one UV source and the at least one UV sensor; calculate the UV radiation dose at one or more locations in the internal space based on the received measurements, for a given starting time; receive an upper UV radiation dose threshold value; and control the at least one UV source to reduce the UV radiation if the maximal calculated UV radiation dose exceeds the upper UV radiation dose threshold value

### CROSS-REFERENCE TO RELATED APPLICATIONS

This application claims the benefit of priority of U.S. Provisional Patent Application No. 63/281,693, titled “SYSTEM AND METHOD FOR UV BASED MICROORGANISM INACTIVATION”, filed Nov. 21, 2021, the contents of which are incorporated herein by reference in their entirety.

### FIELD OF INVENTION

The present invention relates generally to systems and methods for microorganism inactivation. More specifically, the present invention relates to a system and a method for UV-based microorganism inactivation.

### BACKGROUND OF THE INVENTION

Airborne-mediated microbial diseases represent one of the major challenges to worldwide public health. Common examples are influenza, appearing in seasonal and pandemic forms, and bacterially based airborne-mediated diseases such as tuberculosis, increasingly emerging in multi-drug resistant form.

Ultraviolet germicidal irradiation (UVGI) can efficiently kill germs and inactivate both drug-sensitive and multi-drug-resistant bacteria, as well as different strains of viruses. It has several advantages over other inactivation means, for example, UV inactivation is a non-contact method and does not leave traces after its application (except ozone). Until recently, the preferred spectral region for UVGI applications was the UVGI spectral range (250 nm to 280 nm). This spectral range is known to cause human health hazards, being both carcinogenic and cataract genic. Therefore, the use of UVGI in the presence of humans was subject to stringent regulation.

In recent years it was realized that the Far UV-C range of the spectrum (200 nm to 230 nm) is very effective for sterilization, but its health hazard for humans is significantly reduced compared to the UVGI spectral range. Consequently, regulators consider allowing Far UV-C radiation in presence of humans under certain restrictions.

Accordingly, there is a need for an inactivation system based on Far UV-C sources that can be deployed in the presence of humans having the ability to calculate quantitatively the distribution of the radiation field in order to evaluate in real-time the radiation dose delivered to a given point in space or on a surface within an enclosure. The dose value is necessary for the evaluation of the inactivation efficiency and the exposure limits for humans as set by the regulations.

### SUMMARY OF THE INVENTION

Aspects of the invention may be directed to a UV-based microorganism inactivation system, comprising: at least one UV source, configured to emit UV radiation; at least one UV sensor, located at a known location with respect to the at least one UV source; and a controller. In some embodiments, the controller is configured to: receive UV radiation measurements from at least one UV sensor; receive information related to an internal space accommodating the at least one UV source and the at least one UV sensor; calculate the UV radiation dose at one or more locations in the internal space based on the received measurements, for a given starting time; receive an upper UV radiation dose threshold value; and control the at least one UV source to reduce the UV radiation if the maximal calculated UV radiation dose exceeds the upper UV radiation dose threshold value.

In some embodiments, the UV radiation dose is the direct UV radiation dose. In some embodiments, controlling the at least one UV source comprises at least one of limiting an emitting time and reducing an intensity of the UV radiation.

In some embodiments, the controller is further configured to: receive a lower UV radiation dose threshold value; and control at least one UV source to increase the UV radiation if the calculated UV radiation dose is below the lower UV radiation dose threshold value. In some embodiments, controlling at least one UV source comprises at least one of extending an emitting time and increasing the intensity of the UV radiation.

In some embodiments, the internal space comprises a floor and wherein the one or more locations in the internal space are located between one to two meters above the floor. In some embodiments, calculating the UV radiation level at one or more locations comprises: receiving a first location and orientation of the at least one UV source in the internal space; receiving a second location of the at least one UV sensor in the internal space; receiving an angular gain curve and calibration data for the at least one UV sensor; receiving an angular distribution of a UV radiation beam emitted from the at least one UV source; and calculating a UV radiation contribution from each UV source from the at least one UV source at the one or more locations based on the UV radiation-emitting level, the first location, the second location, the sensor angular gain curve, calibration data, and the radiation angular distribution.

In some embodiments, calculating the UV radiation level at the one or more locations comprises calculating at least one of irradiance dose for humans, irradiance dose for surfaces, and fluence dose for air. In some embodiments, the at least one UV source is a Far UV-C source.

In some embodiments, the system further comprises at least one human presence sensor and wherein the controller is further configured to calculate UV radiation dose threshold value based on a detected presence of at least one human. In some embodiments, the detected presence comprises at least one of the locations of one or more humans in the internal space, a trajectory of one or more humans in the internal space, and a number of humans in the internal space.

Some additional aspects of the invention may be directed to a method of controlling a UV based microorganism inactivation system comprising, controlling at least one UV source to emit UV radiation into an internal space; receiving UV radiation measurements from at least one UV sensor; receiving information related to an internal space accommodating the at least one UV source and the at least one UV sensor; calculating a UV radiation dose at one or more locations in the internal space based on the receive measurements for a given starting time; receiving an upper UV radiation dose threshold value; and reducing the UV radiation emitted from the at least one UV source if the calculated UV radiation does exceed the upper UV radiation dose threshold value.

In some embodiments, reducing the UV radiation comprises at least one of limiting an emitting time and reducing an intensity of the UV radiation. In some embodiments, the method may further include, receiving a lower UV radiation dose threshold value; and increasing the UV radiation emitted from the at least one UV source if the calculated UV radiation dose is below the lower UV radiation threshold value.

In some embodiments, increasing the UV radiation comprises at least one of: extending the emitting time and increasing the intensity of the UV radiation. In some embodiments, the internal space comprises a floor and wherein the one or more locations in the internal space are located between one to two meters above the floor.

In some embodiments, calculating the UV radiation dose at the one or more locations comprises: receiving a first location of the at least one UV source in the internal space; receiving a second location of the at least one UV sensor in the internal space; receiving an angular gain curve and calibration data of a UV radiation beam emitted from the at least one UV source; and calculating a UV radiation contribution from each UV source from the at least one UV source at the one or more locations based on the UV radiation-emitting level, the first location, the second location, the angular gain curve, and the calibration data.

In some embodiments, calculating the UV radiation level at the one or more locations comprises calculating at least one of irradiance dose for humans, irradiance dose for surfaces, and fluence dose for air. In some embodiments, the at least one UV source is a Far UV-C source.

In some embodiments, the method further comprises, receiving a detection of human presence from at least one human presence sensor; and calculating the UV radiation dose based on a detected presence of at least one human. In some embodiments, the detected presence comprises at least one of, locations of one or more humans in the internal space, trajectory of one or more humans in the internal space, and a number of humans in the internal space.

Some additional aspects of the invention may be directed to a method of placing one or more UV radiation sources in an internal space, comprising: receiving geometrical information related to an internal space comprising one or more walls; receiving UV radiation-emitting level of each one of the one or more UV sources; determining locations of the one or more UV sources on the one or more walls such that UV radiation variations in a certain volume of interest will not exceed a predetermined value.

In some embodiments, determining the locations of the one or more UV sources comprises calculating the UV radiation does at the one or more locations in the internal space based on the UV radiation-emitting level, the angular gain curve, and the calibration data. In some embodiments, the internal space comprises a floor and wherein the one or more locations in the internal space are located between one to two meters above the floor.

In some embodiments, determining locations of one or more UV sources on the one or more walls is further such that UV radiation doses at one or more locations in the internal space over a first predetermined period of time will not decrease below a first UV radiation dose. In some embodiments, the first UV radiation dose is the minimal UV radiation dose required for the inactivation of at least one type of microorganism.

In some embodiments, determining locations of one or more UV sources on the one or more walls is further such that UV radiation doses at one or more locations in the internal space over a second predetermined period of time will not exceed a second UV radiation dose. In some embodiments, the second given UV radiation dose is the maximal UV dose a human is allowed to be exposed to according to prevailing regulations.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a UV based microorganism inactivation system assembled in an interior space according to some embodiments of the invention;

FIG. 2A is a flowchart of a method of UV based microorganism inactivation according to some embodiments of the invention;

FIG. 2B is a schematic illustration of a UV radiation field in an interior space according to some embodiments of the invention;

FIG. 3 is a schematic illustration of a point UV source emission function according to some embodiments of the invention;

FIG. 4 is a schematic illustration of parameters required for calculating an irradiance of a point source according to some embodiments of the invention;

FIG. 5 is a schematic illustration of parameters required for calculating a fluence of a point source according to some embodiments of the invention;

FIG. 6 is a schematic illustration of a setup for calibrating a UV sensor according to some embodiments of the invention;

FIG. 7 is an illustration of a parallel radiation beam incident on a UV sensor according to some embodiments of the invention;

FIG. 8 is a block diagram, depicting a computing device which may be included in a system for UV based microorganism inactivation according to some embodiments; and

FIG. 9 is a flowchart of a method of placing one or more UV radiation sources in an internal space according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are outlined in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of the same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention disclose a method and a system for UV-based microorganism inactivation. Such a system may include at least one UV source, configured to emit UV radiation and at least one UV sensor, located at a known location with respect to the at least one UV source. In some embodiments, the system may be controlled by a controller configured to, receive UV radiation measurements from the at least one UV sensor; receive information related to an internal space accommodating the at least one UV source and the at least one UV sensor; calculate UV radiation dose at one or more locations in the internal space based on the received measurements, for a given starting time; receive an upper UV radiation dose threshold value; and control the at least one UV source to reduce the UV radiation if the calculated UV radiation dose exceeds the upper UV radiation dose threshold value.

As used herein, radiation dose refers to the amount of radiation energy (measured in energy unit) provided to a specific location. The Radiation dose can be calculated from radiation levels measured at the start and end of the radiation provision. As would be known to one skilled in the art, safety and inactivation levels depend on the radiation dose. The radiation dose in a location is a function of coordinates of the location and possibly the orientation of the radiation.

In some embodiments, the system may be designed to be deployed in an internal space, for example, a room or a venue. The system may reduce the probability of infection in humans present in the treated internal space. The system may cause the inactivation of microorganisms using Far UV-C radiation, by irradiating the internal space in the presence of humans, while imposing the relevant health safety regulations using real-time data. In some embodiments, the system may further include human tracking sensors for tracking the presence of humans.

Table 1 summarizes the reference symbols used herein below.

TABLE 1

| Symbol | Units | Description |
|---|---|---|
| L | W/m²/str | Radiance field. This is the most general |
| | | characterization of the radiation field. The |
| | | irradiance and the fluence fields are derived |
| | | from it. |
| I | W/m² | Irradiance field. The power density incident |
| | | on an area element from a hemisphere. |
| F | W/m² | Fluence field. The power density enters a |
| | | spherical element. |
| D_{I} | J/m² | Irradiance dose. Derived from I by time |
| | | integration over a given period. Can be used to |
| | | estimate health hazards for humans and |
| | | inactivation efficiency on surfaces at a given |
| | | point and orientation. |
| D_{F} | J/m² | Fluence dose. Derived from F by time |
| | | integration over a given period. Can be used to |
| | | estimate inactivation efficiency in the air at |
| | | a given point. |
| R⁽⁰⁾ | 1/str | The gain function. Describes the angular |
| | | dependence of the radiation emitted by a |
| | | UV source. |
| b | W | The total power emitted by a UV source |
| I^{upper bound} | W/m² | The maximum of I taken over a given volume |
| | | and a certain solid angle. Useful for |
| | | time-independent scenarios. |
| I^{lower bound} | W/m² | The minimum of I taken over a given volume |
| | | and a certain solid angle. Useful for |
| | | time-independent scenarios. |
| F^{lower bound} | W/m² | The minimum of F taken over a given |
| | | volume. Useful for time-independent |
| | | scenarios. |
| D_{I}^{upper bound} | J/m² | The maximum of D_{I} taken over a given volume |
| | | and a certain solid angle. Useful for evaluating |
| | | the health hazard within a volume of interest. |
| D_{I}^{lower bound} | J/m² | The minimum of D_{I} taken over a given volume |
| | | and a certain solid angle. Useful for evaluating |
| | | the inactivation efficiency on surfaces within |
| | | a volume of interest. |
| D_{F}^{lower bound} | J/m² | The minimum of D_{F} taken over a given |
| | | volume. Useful for evaluating the inactivation |
| | | efficiency in air within a volume of interest. |

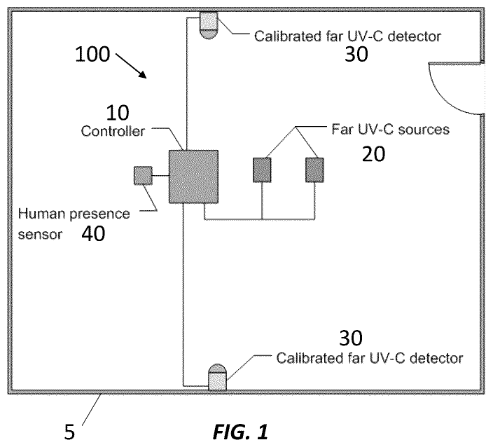

Reference is now made to FIG. 1 which is a schematic illustration of a UV-based microorganism inactivation system assembled in an interior space according to some embodiments of the invention. A system 100 may include, at least one UV source 20, configured to emit UV radiation, at least one UV sensor 30, located at a known location with respect to at least one UV source 20, and a controller 10. In some embodiments, at least one UV source 20 and at least one UV sensor 30 may be assembled at known locations in an internal space 5, for example, on at least one of the walls and/or the sealing of a room, or on an element in the room (e.g., a cabinet, a shelf, etc.). In some embodiments, at least one UV source 20 and/or at least one UV sensor 30 may be assembled on a portable device, and controller 10 may be configured to receive in real-time the current location of at least one UV source 20 and/or at least one UV sensor 30. For example, a portable UV source 20 and/or a portable UV sensor 30 may be attached to a localization sensor configured to provide to controller 10 the current location of UV source 20 and/or UV sensor 30.

In some embodiments, internal space 5 may include a floor and the one or more locations in internal space 5 are located between one to two meters above the floor. Such one or more locations are in a portion of the space (e.g., room) at which humans are expected to breath, thus exhale and inhale microorganisms and viruses. These are the locations in the room that include the air that requires the inactivation and/or inactivation of the microorganisms and viruses.

In some embodiments, at least one UV source 20 may be or may include any type of UV source, for example, far UV-C source. In some embodiments, UV source 20 may emit UV radiation at a constant intensity, and controller 10 may be configured to control the start and end of the UV radiation emission. In some embodiments, UV source 20 may be by an adjustable UV radiation source, and controller 10 also may be configured to control the intensity of the UV emission. In some embodiments, system 100 may include more than one UV source 20, for example, two UV sources 20, as illustrated, three UV sources 20, four UV sources 20, and more. As should be understood by one skilled in the art, the invention is not limited to a specific number of UV sources 20.

In some embodiments, additional information related to at least one UV source 20 may be provided to controller 10, for example, UV radiation-emitting level (e.g., the intensity), a temporal UV radiation-emitting level, an angular distribution of a UV radiation beam emitted from the at least one UV source and the like.

In some embodiments, at least one UV sensor 30 may be or may include any type of detector configured to detect UV radiation. In some embodiments, the number of UV sensors 30 may be equal to or greater than the number of UV sources 20, as discussed hereinbelow.

In some embodiments, controller 10, may be configured to receive extra information in addition to the location of each UV sensor 30. For example, such information may include an angular gain curve (e.g., in the form of a table) and calibration data (e.g., responsivity). In some embodiments, the calibration data may include information to convert the sensor output (Amperes) to irradiance (Watts/m²). A particular calibration process is disclosed herein below with respect to equations 19-22.

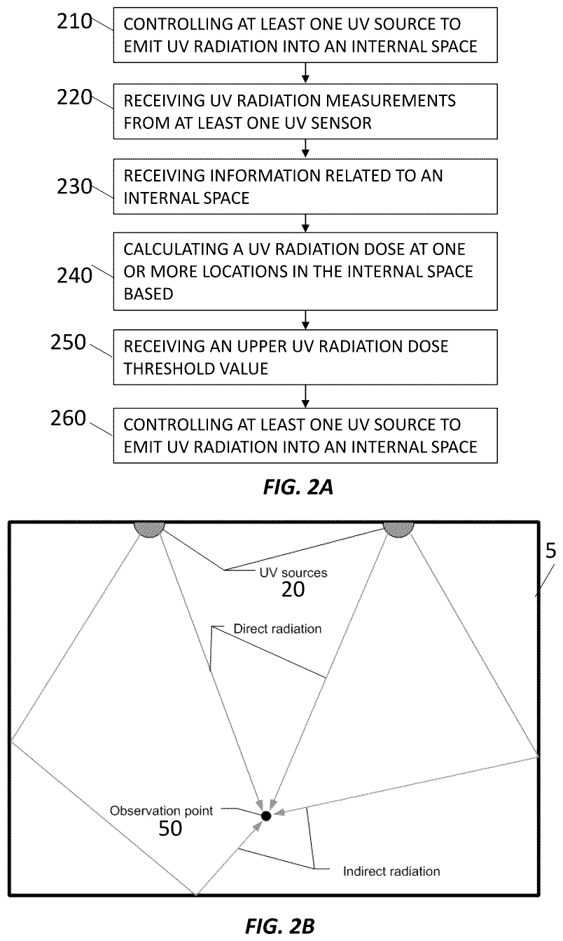

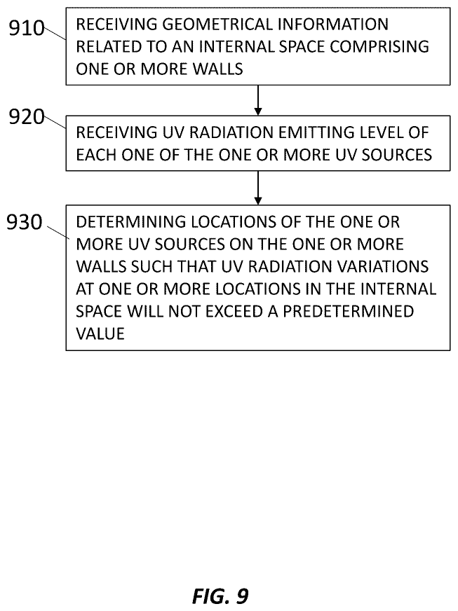

Controller 10 may include any computing device, for example, the computing device illustrated and discussed with respect to FIG. 8 herein below. Controller 10 may be configured to execute methods according to embodiments of the invention, for example, the methods of FIG. 2A and FIG. 9.

In some embodiments, system 100 may further include one or more human presence sensors 40. For example, human presence sensors 40 may be selected from, proximity sensors, a space detector, a movement detector, a camera, and the like. In some embodiments, one or more human presence sensors 40 may be configured to detect at least one of the locations of one or more humans in the internal space, a trajectory of one or more humans in the internal space, and a number of humans in the internal space.

FIG. 2A is a flowchart of a method of UV-based microorganism inactivation according to some embodiments of the invention. The method of FIG. 2A may be executed by any controller, for example, controller 10.

In step 210, controller 10 controls at least one UV source to emit UV radiation into an internal space. In some embodiments, controller 10 may control at least one UV source 20 to start emitting UV radiation. In some embodiments, if UV source 20 is an adjustable UV source, controller 10 may further control the intensity of the UV radiation.

In step 220, controller 10 receives UV radiation measurements from at least one UV sensor 30. In some embodiments, controller 10 may receive from each sensor 30, the start and end time at which an increase (above background level) of the intensity of the UV radiation was detected and the detected intensity. Controller 10 may further receive the location of each UV radiation source 30 at internal space 5 and an angular gain curve and calibration data for each UV radiation sensor 30.

In step 230, controller 10 may receive information related to internal space 5 accommodating the at least one UV source and the at least one UV sensor. For example, controller 10 may receive the dimensions of internal space 5, the locations (e.g., coordinates) of each one of UV sensors 30, and UV sources 20.

In step 240, controller 10 may be configured to calculate a UV radiation dose at one or more locations in the internal space based on the received measurements for a given starting time.

In some embodiments, calculating the UV radiation dose may include calculating the UV radiation field in internal space 5, as illustrated in FIG. 2B.

UV radiation can be characterized by at least one of four characterizations: radiance, irradiance, fluence, and dose. Radiance, irradiance, fluence are field power density characterizations and dose characterizes radiation energy density.

### Radiance

The radiance field L is used to compute the power dP incident on an area element dA forming a solid angle dΩ around a direction k, according to equation (1).

$$dP ( x , k , n ) = L ( x , k ) cos θ dAd Ω , \qquad ( 1 )$$

where θ is the angle between the normal n to the surface element and k. The radiance field is measured in units of W/m²/str. It depends on the position x the direction k, and the normal direction n, shown for example, in FIG. 4. The radiance field may be time-dependent making all quantities derived from it time-dependent too. In the description below, the time argument is suppressed when it is irrelevant as in equation 1 for the sake of simplicity.

### Irradiance

The irradiance field I is the total power density flowing through a surface element per unit area. It is related to the radiance according to equation (2).

$$I ( x , n ) = \int \int L ( x , k ) cos θ d Ω ( k ) . \qquad ( 2 )$$

The integration is carried over a hemisphere (relative to the surface element). Therefore, it represents the power incident on the surface element from one side only. The radiance field is measured in units of W/m². The irradiance field depends on the position x and the direction of the normal n of the surface element.

If the radiance field L is isotropic (independent of the direction), then according to equation (2).

$$I ( x ) = π L ( x ) . \qquad ( 3 )$$

This is characteristic of a radiation field in thermal equilibrium.

### Fluence

The fluence field F is the total radiation power dP entering an infinitesimal sphere of radius dr divided by the sphere's equatorial section area, calculated from equation (4).

$$F ( x ) = \frac{dP}{π \cdot {dr}^{2}} . \qquad ( 4 )$$

The fluence depends on the position x only. If the radiance field is isotropic, then according to equation (4),

$$F ( x ) = 4 π L ( x ) \qquad ( 5 )$$

### Dose

The dose characterizes radiation energy density and is obtained by integrating the power with respect to time for a given time interval [t₀, t]. One can define dose for each one of the two field power characterizations, irradiance, and fluence. The corresponding doses are measured in units of J/m₂.

The relevant dose depends on whether the irradiated object is a surface or a point in space. For a point in space, the relevant dose is the fluence time integrated from a certain initial time t₀ to t, which is given by equation (6),

$${D}_{F} ( x , t ) = {{\int }_{{t}_{0}}}^{t} F ( x , {t}^{′} ) {dt}^{′} . \qquad ( 6 )$$

Lethal dose is the dose which achieves a certain satisfactory level of inactivation. Different organisms have different values of lethal dose. Equation (6) may be used for calculating the required dose to be applied to planktonic (free-floating) organisms, which are exposed to the UV radiation from the whole surrounding sphere. However, if the microorganisms are protected by mechanical particles, such as dust and dirt, or have formed biofilm, a much higher UV fluence may be needed to achieve the required level of inactivation.

If the irradiated object is a surface, the relevant dose is the irradiance dose. The irradiance dose can be calculated using equation (7),

$${D}_{I} ( x , n , t ) = {{\int }_{{t}_{0}}}^{t} I ( x , n , {t}^{′} ) {dt}^{′} \qquad ( 7 )$$

The irradiance dose depends on the surface normal direction n, whereas the fluence dose is independent of any direction.

### Mapping the UV Radiation Fields

The above equations can be used for mapping the UV radiation field, as illustrated in FIG. 2B. The UV radiation field may be generated by one or more UV radiation sources (e.g., sources 20), which may be located on the boundaries of the interior space, for example, at a room's ceiling, as illustrated. The UV radiation field has two components, direct and scattered. The direct radiation can be calculated from the equations presented below. The scattered irradiation is more difficult to calculate. Typically, for the Far UV-C spectral region the material reflection coefficients are usually small, so, in the presence of direct fields, the contribution of the scattered fields can be usually neglected.

In some embodiments, the scattered irradiation or indirect irradiation may be taken into consideration, for example, when one or more mirrors are located on one or more walls of the interior space. In such radiation the reflected back from the one or more mirrors can be calculated based on the location/size of the mirror with respect to each one of the radiation sources.

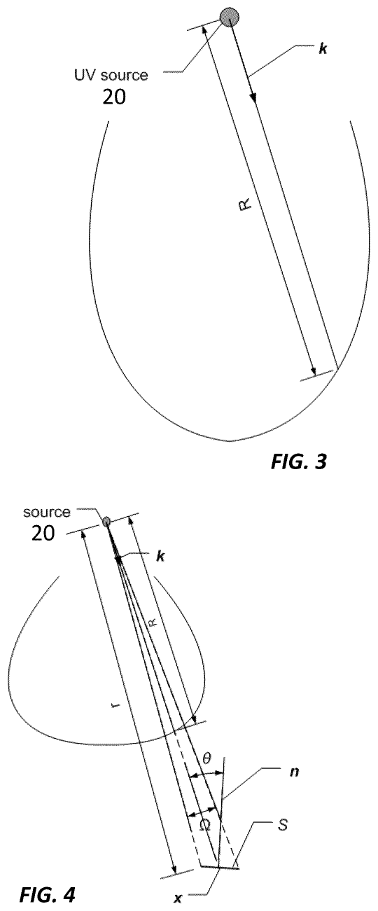

Reference is now made to FIG. 3 which is an illustration of UV emission form a point source, according to some embodiments of the invention. The radiation field of a point source is characterized by an emission function R, which is measured in units of W/str. The power P emitted into a solid angle dΩ around a direction k is given by equation (8).

$$P = R ( k ) d Ω \qquad ( 8 )$$

The values of the emission function are usually measured experimentally. The length of the line denoted by R in FIG. 3, is proportional to the value of the emission function in that direction.

The solid angle dΩ subtended by a surface element S with respect to the point x is given by equation (9).

$$d Ω = \frac{S cos θ}{{r}^{2} ( x )} \qquad ( 9 )$$

The various terms and variables of equation (9) are illustrated in FIG. 4. The power P incident on element S may be calculated from equation (10) which is derived with the help of equations 8 and 9.

$$P ( x , n ) = \frac{R [ k ( x ) ] S cos θ}{{r}^{2} ( x )} \qquad ( 10 )$$

The irradiance is the power divided by S and can be calculated from equation 11.

$$I ( x , n ) = \frac{R [ k ( x ) ] S cos θ}{{r}^{2} ( x )} \qquad ( 11 )$$

In some embodiments, contribution to the irradiance from two or more UV point radiation sources 20 is calculated by summing over all individual irradiance values from each source, using equation 12.

$$I ( x , n ) = {\sum }_{n} \frac{{R}_{n} [ {k}_{n} ( x ) ] cos {θ}_{n}}{{{r}_{n}}^{2} ( x )} \qquad ( 12 )$$

where Rₙ is the emission function of the n-th source, kₙ is the direction to the n-th source, θₙ is the angle between kₙ and the normal to the surface element, and rₙ is the distance from the surface element to the source.

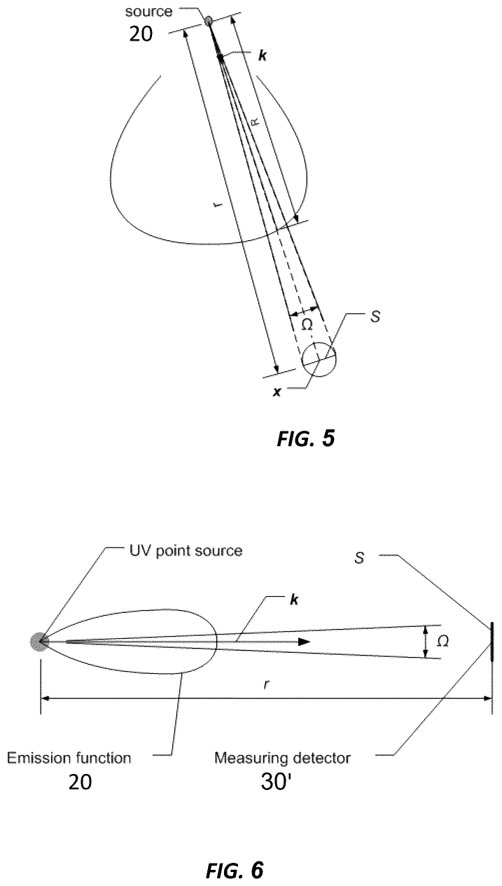

For the calculation of the fluence, a small sphere is imagined at a distance r from the source, as illustrated in FIG. 5. The surface element for the calculation is an equatorial section of this sphere, with its normal parallel to the direction of the source k (also shown in FIG. 5). In such case the angle θ becomes zero, so the corresponding solid angle becomes as stated in equation 13.

$$Ω = \frac{S}{{r}^{2} ( x )} \qquad ( 13 )$$

Therefore, the power P incident on the element S can be derived from equation 8, to produce equation 14,

$$P ( x ) = \frac{R [ k ( s ) ] S}{{r}^{2} ( x )} \qquad ( 14 )$$

and since the fluence is the power divided by S, the fluence can be calculated using equation 15.

$$F ( x ) = \frac{R [ k ( x ) ]}{{r}^{2} ( x )} \qquad ( 15 )$$

In some embodiments, contribution to the fluence from two or more UV point radiation sources 20 is calculated by summing over all individual fluence values from each source, using equation 16.

$$F ( x ) = {\sum }_{n} \frac{{R}_{n} [ {k}_{n} ( x ) ]}{{{r}_{n}}^{2} ( x )} \qquad ( 16 )$$

where Rₙ is the emission function of the n^{th} source, kₙ is the direction to the n^{th} source, and rₙ is the distance to it.

In some embodiments, UV radiation sources 20 which cannot be approximated by a point are called “extended”. An extended source may be regarded as a collection of point sources. The fields of extended sources can be calculated by summing the contributions of the point sources which make up the extended source, using equations (11) and (16).

### Mapping the UV Radiation Field Based on Measurements from Multiple UV Sensors

In some embodiments, the emittance function R of a given source is changing with time. Two main mechanisms contribute to this change, reduction in the source UV generation efficiency, and deposit of dust on the source reflector. In some embodiments, using the lowest approximation, the first mechanism affects the overall amplitude of the emission function, but not its geometry. In this approximation, the time-dependent emittance function can be written using equation 17.

$$R ( k , t ) = b ( t ) {R}^{( 0 )} ( k ) \qquad ( 17 )$$

For simplicity and convenience, units of power (Watts) were assigned to b(t). In this case, R⁽⁰⁾(k) will have units of 1/str and will be referred to as “gain function”.

The gain function is normalized so that

$$\int \int {R}^{( 0 )} ( k ) d Ω ( k ) = 1$$

where the integration is carried over all directions k and dW is an infinitesimal solid angle around k. With this normalization, function b(t) will be the total UV power emitted by the source.

Under the assumption implied by equation 17, the radiation fields (irradiance and fluence) in a given point x are given by equations 18 and 19.

$$I ( x , n ) = {\sum }_{n = 1}^{N} \frac{{b}_{n} {{R}_{n}}^{( 0 )} [ {k}_{n} ( x ) ] cos [ θ ( n ) ]}{{{r}_{n}}^{2} ( x )} \qquad ( 18 )$$

$$and$$

$$F ( x ) = {\sum }_{n = 1}^{N} \frac{{b}_{n} {{R}_{n}}^{( 0 )} [ {k}_{n} ( x ) ]}{{{r}_{n}}^{2} ( x )} \qquad ( 19 )$$

Both the irradiance and fluence are dependent on the coefficients bₙ.

In some embodiments, the value of b for each UV sources 20 may be determined from the output of the dedicated sensors. In some embodiments, this requires the calibration of these sensors. The calibration can be done by measuring the radiation power flux (W/m²) at a given distance from the source with an irradiance measurement device 30′ (as illustrated in FIG. 6). The radiation should be normal to the irradiance measurement device, as illustrated. The power reaching the irradiance measurement device 30′ can be calculated using equation 8 and is given by equation 20.

$$P = {bR}^{( 0 )} ( k ) Ω , \qquad ( 20 )$$

where

$$Ω = \frac{S}{{r}^{2}} .$$

In some embodiments, power flux U is therefore given by equation 21.

$$U = \frac{{bR}^{( 0 )} ( k )}{{r}^{2}} . \qquad ( 21 )$$

Therefore, the value of b can be calculated from equation 22.

$$b = \frac{{r}^{2} U}{{R}^{( 0 )} ( k )} \qquad ( 22 )$$

Accordingly, a calibration factor q for the dedicated sensor can be calculated from equation 23.

$$q = \frac{b}{i} , \qquad ( 23 )$$

where i is the dedicated sensor output current.

In some embodiments, dedicated sensors can be added to all radiation sources and calibrated as explained above. By finding a corresponding bₙ value for each UV radiation source 20, the irradiance and the fluence fields can be calculated from equations 18 and 19.

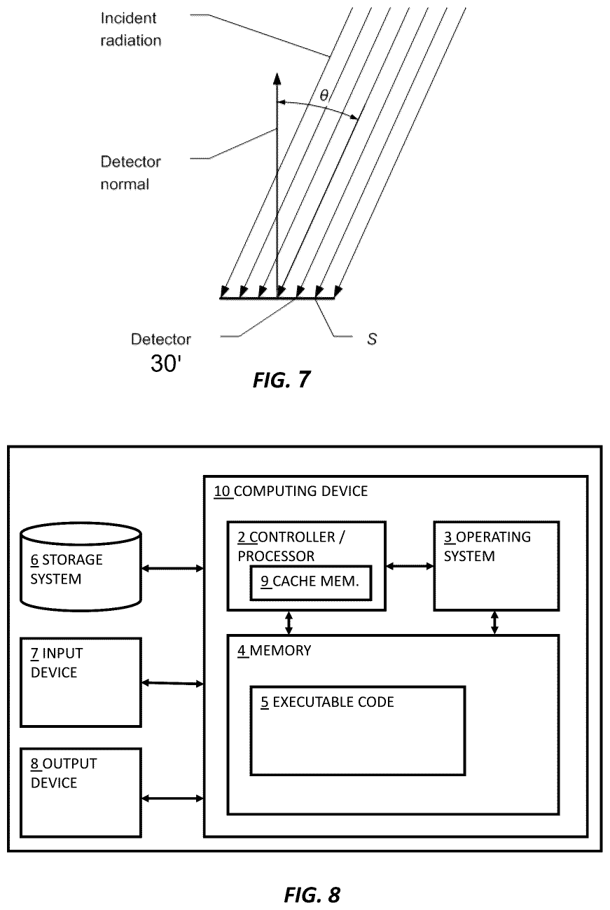

In some embodiments, the value bₙ for each radiation source 20 can be calculated from measurements received from a remote sensor. FIG. 7 is an illustration of a scheme of a parallel beam incident on a sensor according to some embodiments of the invention.

In some embodiments, the response of a sensor depends on the angle of the incident radiation with respect to its surface normal. The output current of a sensor irradiated by a radiation incident at an angle θ can be written using equation 24.

$${i}_{d} = η \cdot U \cdot S \cdot d ( θ ) \qquad ( 24 )$$

The various quantities appearing in equation 24 are listed in Table 2.

TABLE 2

| Symbol | Description | Units |
|---|---|---|
| i_{d} | Detector output current | Amperes |
| h | Detector responsivity | Amperes/Watt |
| U | Incident power density | Watt/m² |
| S | Detector area | m² |
| q | Angle between incoming radiation and normal | — |
| d(θ) | Detector's angular responsivity | — |

The sensor angular responsivity is defined up to a multiplicative constant. It is convenient to set this constant by requiring that d(0)=1.

The UV irradiance for a point source can be calculated from equation 11. Therefore, i_{d} can be cast from equation 25.

$${i}_{d} = η \frac{{bR}^{( 0 )} [ k ( x ) ] cos θ}{{r}^{2} ( x )} Sd ( θ ) \qquad ( 25 )$$

Therefore, the value of b can be derived from the detector current output i_{d}.

In some embodiments, the values bₙ can be measured for multiple UV sources using several UV sensors. When a sensor is exposed to several sources, the power incident on its surface is the sum of all contributions of the different sources. In such case, one will use several sensors 30 disposed at different locations of the enclosure to measure the b values of the sources. The output of the m′th detector can be written as a set of equations 26.

$${{i}_{d}}^{m} = η S {\sum }_{n = 1}^{N} \frac{{b}_{n} {{R}_{n}}^{( 0 )} [ {k}_{n} ( {x}_{m} ) ] cos [ {θ}_{n} ( {n}_{m} ) ] d [ {θ}_{n} ( {n}_{m} ) ]}{{{r}_{n}}^{2} ( {x}_{m} )} , m = 1 , ... , K \qquad ( 26 )$$

where N and K are the number of sources and sensors respectively. Set 26 a set of linear equations which can be solved for the bₙ values if K≥N. Accordingly, in order to derive the bₙ values from measurements taken from remote sensors, the number of sensors 30 in system 100 must be larger than or equal to the number of UV sources 20.

In some embodiments, the system may comprise several UV sources, some having dedicated sensors and some not. In such embodiments, the bₙ coefficients of the UV sources with dedicated sensors should be treated as known in the system of equations 26. Denoting the number of sources with dedicated sensors by M, the number of unknowns in the system of equations 26 becomes N−M. In such case, the number of sensors K must exceed N−M to allow the computation of the unknown bₙ coefficients.

In some embodiments, once the values of all the bₙ coefficients are determined, either with dedicated or remote detectors, the values of the UV field (irradiance and fluence) at any point within the enclosure can be calculated from equations 18 and 19.

### Calculating the UV Dose

Referencing back to FIG. 2A and step 240, the UV dose may be calculated with respect to a given interval of time [t₀, t], where t₀ and t are the start and the end times respectively of the radiation inspection. This time interval is referred herein as the “inspection period”.

There are at least three types of doses that are of interest according to some embodiments of the invention: the irradiance dose for surfaces D_{I} (equation 7), and the fluence dose for air D_{F} (equation 6). If a human tracking device is available, an additional dose can be of interest: the tracking dose D_{tracking}.

In some embodiments, there is a need to guarantee system performance in a certain volume within the enclosure and not just at a certain point in air or on a surface. In such embodiments, it is useful to introduce upper and lower bounds for the irradiance field, as given in equations 29.

$${{D}_{I}}^{upper bound} ( t ) = {max}_{n , x} \{ {D}_{I} ( x , n , t ) \} \qquad ( 29 )$$

$${{D}_{I}}^{lower bound} ( t ) = {min}_{n , x} \{ {D}_{I} ( x , n , t ) \}$$

where the maximization or the minimization is carried over both the normal orientation n and the position x. The operations with respect to x are carried over a certain volume of interest, and over n within a certain solid angle of interest. The upper bound of the irradiance dose may be useful for the evaluation of the health hazard, and the lower bound for the evaluation of the inactivation degree.

In such embodiments, the lower bound of the fluence dose (equation 6) may be used to estimate the inactivation efficiency:

$${{D}_{F}}^{lower bound} ( t ) = {min}_{x} \{ {D}_{F} ( x , t ) \} \qquad ( 30 )$$

where the minimization is carried over a certain volume of interest.

In such embodiments, D_{I}^{lower bound}(t) may be used to estimate the inactivation efficiency on surfaces within a volume of interest, and D_{I}^{upper bound}(t) may be used to evaluate the health hazard for humans within the volume of interest.

In some embodiments, the radiance field L is independent of time within the inspection period, so both the irradiance and the fluence fields are time-independent too. In such case, the fluence dose (equation 6) is given by

$${D}_{F} ( x , t ) = F ( x ) t \qquad ( 31 )$$

where the initial time t₀ was set to 0 for simplicity (and without loss of generality). Correspondingly, the irradiance dose (equation 7) will be given by

$${D}_{I} ( x , n , t ) = I ( x , n ) t \qquad ( 32 )$$

In such embodiments, it is useful to introduce upper and lower values for the irradiance and the fluence fields:

$${I}^{upper bound} = {max}_{n , x} \{ I ( x , n ) \} \qquad ( 35 )$$

$${I}^{lower bound} = {min}_{n , x} \{ I ( x , n ) \} \qquad ( 36 )$$

$${F}^{lower bound} = {min}_{x} \{ F ( x ) \} \qquad ( 37 )$$

In such case, the corresponding doses are given by

$${{D}_{I}}^{upper bound} ( t ) = {I}^{upper bound} \cdot t \qquad ( 38 )$$

$${{D}_{I}}^{lower bound} ( t ) = {I}^{lower bound} \cdot t \qquad ( 39 )$$

$${{D}_{F}}^{lower bound} ( t ) = {F}^{lower bound} \cdot t \qquad ( 40 )$$

Let us denote by D_{I}^{r} the maximal dose allowed for humans as defined by the regulations, and by D_{I}ⁱ the lethal dose required for the inactivation of the micro-organisms of interest. In such case, the maximal exposure time t_{M} allowed by regulations is derived from equation 38 and given by equation 38:

$${t}_{M} = \frac{{{D}_{I}}^{r}}{{I}^{upper bound}} \qquad ( 41 )$$

Accordingly, the minimal time tₘ^{s} required for inactivation on surfaces can be derived from equation 39:

$${{t}_{m}}^{s} = \frac{{{D}_{I}}^{i}}{{I}^{lower bound}} \qquad ( 42 )$$

Furthermore, the minimal time tₘ^{a} required for inactivation of micro-organisms in air can be derived from equation 40:

$${{t}_{m}}^{a} = \frac{{{D}_{F}}^{i}}{{F}^{lower bound}} \qquad ( 43 )$$

where D_{F}ⁱ is the fluence dose required for inactivation of the micro-organisms in air.

The following two ratios are of interest in some practical embodiments:

$${r}_{s} = \frac{{t}_{M}}{{{t}_{m}}^{s}} = \frac{{{D}_{h}}^{r}}{{{D}_{h}}^{i}} \frac{{I}^{lower bound}}{{I}^{upper bound}} \qquad ( 44 )$$

$$and$$

$${r}_{a} = \frac{{t}_{M}}{{{t}_{m}}^{a}} = \frac{{{D}_{h}}^{r}}{{{D}_{F}}^{i}} \frac{{F}^{lower bound}}{{I}^{upper bound}} \qquad ( 45 )$$

Denoting the variation of the irradiance field (over the volume and solid angle of interest) by 2ΔI, and its average by Ī. In such case rₛ can be written as

$${r}_{s} = \frac{{t}_{M}}{{{t}_{m}}^{s}} = \frac{{{D}_{h}}^{r}}{{{D}_{h}}^{i}} \frac{\overline{I} - Δ I}{\overline{I} + Δ I} \qquad ( 46 )$$

If rₛ is larger than 1, then inactivation on surfaces can be achieved before the human health hazard dose is reached. If rₛ is smaller than 1, then the light sources will have to be shut down before the inactivation dose on surfaces is reached. In such case, inactivation on surfaces cannot be achieved with human presence.

Let us denote the variation of the irradiance field (over the volume of interest) by 2ΔF, and its average by F̅. In such case rₛ can be written as

$${r}_{a} = \frac{{t}_{M}}{{{t}_{m}}^{a}} = \frac{{{D}_{h}}^{r}}{{{D}_{F}}^{i}} \frac{\overline{F} - Δ F}{\overline{I} + Δ I} \qquad ( 47 )$$

If rₐ is larger than 1, then inactivation in air can be achieved before the human health hazard dose is reached. If rₐ is smaller than 1, then the light sources will have to be shut down before the inactivation dose in air is reached. In such case, inactivation in air cannot be achieved with human presence.

To allow effective inactivation while avoiding health hazards by safe margins, the ratios rₛ and rₐ must be as large as possible. Both are products of two factors (equations 46 and 47). The first factors in both are derived from scientific research and the system designer cannot modify their values. On the other hand, the second factors reflect the radiation field characteristics and can be controlled by system design. It can be appreciated that the values of both rₛ and rₐ are maximized if the field variations ΔI and ΔF are minimized.

The value of rₛ reaches its maximum value for a given average irradiance Ī if the irradiance field variations ΔI are designed to vanish.

Typically, when the irradiance field variations ΔI vanish, the fluence field variations ΔF vanish too. In such case, also rₐ reaches its maximum if the field variations ΔI is designed to vanish.

These arguments explain why in practical embodiments the system performance can be optimized by designing the UV sources layout to minimize the irradiance field variations within a volume and solid angle of interest.

In some embodiments, a detection of human presence may be received form at least one human presence sensor 40. In some embodiments, human presence sensor 40 may be, a human tracking device (e.g., a camera, movement detector, information received from a user's mobile device carried by the user, or any other tracking device carried by the human) configured to track a human in internal space 5. In some embodiments, human presence sensor 40 may be a human presence device, for example, a space detector or the like.

In some embodiments, a human presence device may be used for determining the inspection period for irradiance dose. Assuming that sensor 40 can only detect the presence of humans, therefore may provide the total time interval [t₀, t] that a human was present in the space 5 within the inspection time interval.

In some embodiments, the human tracking device may provide a trajectory x(t) for each human in the enclosure. Since such device does not supply information on the orientation of the human surfaces that are exposed to the UV radiation, one must use instead upper and lower bound estimates. Therefore, the upper and the lower bounds of the tracking irradiance dose I for a given human characterized by a trajectory x(t) provided by sensor 40 can be calculated using equations 27.

$${{I}_{tracking}}^{upper bound} ( t ) = {max}_{n} \{ I ( x ( t ) , n ) \} \qquad ( 27 )$$

$${{I}_{tracking}}^{lower bound} ( t ) = {min}_{n} \{ I ( x ( t ) , n ) \}$$

where I(x,n) is given by equation 18 and the maximum or the minimum is taken over orientations of the normal n characterizing all normal directions of the exposed surfaces of the humans of interest.

In some embodiments, the upper and the lower bounds of the tracking dose may be determined now from equations 28.

$${{D}_{tracking}}^{upper bound} = {{\int }_{{t}_{0}}}^{t} {{I}_{tracking}}^{upper bound} ( {t}^{′} ) {dt}^{′} \qquad ( 28 )$$

$${{D}_{tracking}}^{lower bound} = {{\int }_{{t}_{0}}}^{t} {{I}_{tracking}}^{lower bound} ( {t}^{′} ) {dt}^{′}$$

If the trajectories x(t) are known, it is possible to use D_{tracking}^{upper bound} instead of D_{I}^{upper bound} for the health hazard estimation for any given human. Similarly, it is possible it is possible to use D_{tracking}^{lower bound} instead of D_{I}^{lower bound} for the human health hazard estimation.

In some embodiments, controller 10 may set starting time t₀ as the initial detection of the presence of a human, by one or more sensors 40. Additionally or alternatively, the human presence may be provided to controller 10 by the user, for example, using a user device indicating that the user entered a room (e.g., using a control box on the rooms walls) and t₀ may be the time the user first used the control box. In some embodiments, t may be set by controller 10 at any time interval required to check the UV dose to which the user was exposed to. For example, t may be set 2 minutes after t₀. In some embodiments, controller 10 may calculate the dose at predetermined time intervals, for example, time intervals of 1, 2, 3, 4, 5, 6, 10 minutes or more.

In some embodiments, after setting t₀ and t the controller 10 may calculate the dose using any one or all of equations 27-32.

In step 250, controller 10 may receive an upper UV radiation dose threshold value. The upper UV radiation dose threshold values may be received from standardizations and regulations set by research, regulatory, and health organizations, for example, the International Ultraviolet Association (IUVA)

In step 260, controller 10 may reduce the UV radiation emitted from at least one UV source 20 if the calculated UV radiation does exceed the upper UV radiation dose threshold value. For example, controller 10 may limit an emitting time, by shutting done one or more UV sources 20. In another example, when one or more UV sources 20 are adjustable sources, controller 10 may reduce an intensity of the UV radiation for at least one UV source 20.

In some embodiments, the method may further include receiving another UV radiation dose threshold value, for example, the inactivation threshold value. The inactivation threshold value may be compared to D_{I} and D_{F}. If spatial information is not of interest, one can use the lower bounds Dₕ^{lower bound} and D_{F}^{lower bound} instead (equation 29, 30).

In some embodiments, if the calculated D_{I} and D_{F}, or the corresponding lower bounds, for a given t₀ and t, result to be equal to or above the inactivation threshold value, controller 10 may shout down all the UV sources 20. In some embodiments, if the calculated UV radiation dose is below the lower UV radiation dose threshold value, controller 10 may increasing the UV radiation emitted from the at least one UV source, for example, by either increasing the emission duration and/or increasing the emission intensity.

In some embodiments, calculating the UV radiation dose at the one or more locations x may include, receiving a first location of the at least one UV source in the internal space; receiving a second location of the at least one UV sensor in the internal space; receiving an angular gain curve (e.g., any one of the gain curves illustrated at FIGS. 5, 6 and 7 and the corresponding equations) and calibration data (e.g., bₙ and q) of a UV radiation beam emitted from the at least one UV source; and calculating a UV radiation contribution from each UV source from the at least one UV source at the one or more locations based on the UV radiation emitting level, the first location, the second location, the angular gain curve and the calibration data, for example, using any one or all of equations 27-32.

Reference is now made to FIG. 8, which is a block diagram depicting a computing device, which may be included within an embodiment of a UV based microorganism inactivation system, according to some embodiments.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 10 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may control UV based microorganism inactivation as further described herein. Although, for the sake of clarity, a single item of executable code 5 is shown in FIG. 8, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Information related to an internal space accommodating at least one UV source and at least one UV sensor in storage system 6 and may be loaded from storage system 6 into memory 4 where it may be processed by processor or controller 2. In some embodiments, some of the components shown in FIG. 8 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 10 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 10 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

### Designing the System

In some embodiments, designing a system such as system 100 to be assembled in an internal space 5, may require determining the number and locations of UV radiation sources 20, the number and locations of UV radiation sensors 30 and the requirement or the number and locations of human presence sensors 40.

Reference is now made to FIG. 9, which, which is a flowchart of a method of placing one or more UV radiation sources in an internal space according to some embodiments of the invention. Using the method of FIG. 9 may allow designing UV based microorganism inactivation system for specific internal spaces, for example, emergency rooms, operation rooms, doctor's office, medical clinics, etc.

In step 910, geometrical information related to an internal space may be received. In some embodiments, the internal space may include one or more walls, sealing, one or more fixed element (e.g., a cabinet, table, etc) and a floor, for example, internal space 5 may be a room.

In step 920, UV radiation emitting level may be received of each one of the one or more UV sources. For example, several types of UVC sources 20 may be considered, each having different emitting intensity. In some embodiments, adjustable UV sources may also be considered.

In step 930, locations of the one or more UV sources on the one or more walls, ceiling and/or an element in the internal space (e.g., space 5), may be determined so that variations of irradiance or fluence values at one or more locations in the internal space will not exceed a predetermined value.

In some embodiments, locations of the one or more UV radiation sensors 30 on the one or more walls, ceiling and/or an element in space 5 may also be determined.

In some embodiments, an angular gain curve and calibration data for a UV radiation beam may be received for each one of the one or more UV sources. For example, angular gain curve such as any one of the gain curves illustrated at FIGS. 5, 6 and 7 and the corresponding equations) and calibration data comprising b, and q may be received for each one or more UV sources 20.

In some embodiments, the locations and/or orientations of the one or more UV sources may be determined by calculating UV radiation dose at the one or more locations and/or orientations in the internal space based on the UV radiation emitting level, the angular gain curve and the calibration data. For example, controller such as controller 10 may calculate UV radiation does using any one or all of equations 27-32. In some embodiments, the location of the one or more UV sources may be determined to provide an optimized UV radiation to locations in internal space 5, being at one to two meters from a floor of internal space 5.

In some embodiments, determining locations of one or more UV sources on the one or more walls is further such that UV radiation doses at one or more locations in the internal space over a first predetermined period of time will not decrease below a first UV radiation dose. In some embodiments, the first UV radiation dose is the minimal UV radiation dose required for the inactivation of at least one type of microorganism.

In some embodiments, determining locations of one or more UV sources on the one or more walls is further such that UV radiation doses at one or more locations in the internal space over a second predetermined period of time will not exceed a second UV radiation dose. In some embodiments, the second given UV radiation dose is the maximal UV dose a human is allowed to be exposed to according to prevailing regulations.

In some embodiments, the orientations of at least some of UV sources 20 may further be determined. The orientation comprises the direction of the central axis of the UV radiation function (indicated as an arrow in FIG. 6). In some embodiments, at least some of UV sources 20 may be mounted on a three-angle adjustable stage configured to move of the central axis to any direction inside internal space 5 and to rotate the UV sources around this axis by any given angle, and controller 10 may further be configured to control the three-angle adjustable stage, as to optimized the provision of UV radiation to internal space 5.

In some embodiments, the locations and the orientations of one or more UV sources are determined such that differences in the UV radiation levels in a certain volume of interest (e.g., between one to two meters form the floor of space 5) in the internal space is not exceeding a certain small value. In such case the radiation within the volume of interest may be substantially homogeneous.

In some embodiment, determining locations of one or more UV sources on the one or more walls, sealing and/or an element in space 5, is further such that UV radiation doses at one or more locations in the internal space will not decrease below a lower UV radiation dose threshold value. In some embodiments, the lower UV radiation dose threshold value is the minimal UV radiation level dose required for inactivation of at least one type of microorganism over a first predetermined period of time.

Accordingly, the disclosed system and methods provides a solution for UV based microorganism inactivation of large internal spaces, such as, rooms, in the presence of humans. The disclosed system and methods may allow to inactivate microorganism in the air and one surface within the internal spaces, for example, at locations in a portion of the space (e.g., room) at which humans are expected to breath, thus exhale and inhale microorganisms and viruses.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A UV based microorganism inactivation system, comprising:
at least one UV source, configured to emit UV radiation;
at least one UV sensor, located at a known location with respect to the at least one UV source; and
a controller configured to:
receive UV radiation measurements from the at least one UV sensor;
receive information related to an internal space accommodating the at least one UV source and the at least one UV sensor;
calculate the UV radiation dose at one or more locations in the internal space based on the received measurements, for a given starting time;
receive an upper UV radiation dose threshold value; and
control the at least one UV source based on the calculated UV radiation dose and based on the upper UV radiation dose threshold value.

2. The system of claim 1, wherein the UV radiation dose is a direct UV radiation dose.

3. The system of claim 1, wherein controlling the at least one UV source comprises at least one of: limiting an emitting time and reducing an intensity of the UV radiation.

4. The system of claim 1, wherein the controller is further configured to:
receive a lower UV radiation dose threshold value; and
control the at least one UV source also based on the lower UV radiation dose threshold value.

5. The system of claim 3, wherein controlling the at least one UV source comprises at least one of: extending an emitting time and increasing an intensity of the UV radiation.

6. The system according to claim 1, wherein the internal space comprises a floor and wherein the one or more locations in the internal space are located between one to two meters above the floor.

7. The system according to claim 1, wherein calculating the UV radiation level at the one or more locations comprises:
receiving a first location and orientation of the at least one UV source in the internal space;
receiving a second location of the at least one UV sensor in the internal space;
receiving an angular gain curve and calibration data for the at least one UV sensor;
receiving an angular distribution of a UV radiation beam emitted from the at least one UV source; and
calculating a UV radiation contribution from each UV source from the at least one UV source at the one or more locations based on the UV radiation emitting level, the first location, the second location, the sensor angular gain curve, calibration data, and the radiation angular distribution.

8. The system according to claim 1, wherein calculating the UV radiation level at the one or more locations comprises calculating at least one of: irradiance tracking dose, irradiance dose for humans, irradiance dose for surfaces, and fluence dose for air.

9. The system according to claim 1, wherein the at least one UV source is a Far UV-C source.

10. The system according to claim 1, further comprising at least one human presence sensor and wherein the controller is further configured to calculate UV radiation dose threshold value based on a detected presence of at least one human.

11. (canceled)

12. A method of controlling a UV based microorganism inactivation system comprising:
controlling at least one UV source to emit UV radiation into an internal space;
receiving UV radiation measurements from at least one UV sensor;
receiving information related to an internal space accommodating the at least one UV source and the at least one UV sensor;
calculating a UV radiation dose at one or more locations in the internal space based on the receive measurements for a given starting time;
receiving an upper UV radiation dose threshold value; and
controlling the UV radiation emitted from the at least one UV source based on the calculated UV radiation dose and based on the upper UV radiation dose threshold value.

13. The method of claim 12, wherein controlling the UV radiation comprises at least one of: limiting an emitting time and reducing an intensity of the UV radiation.

14. The method of claim 12, further comprising:
receiving a lower UV radiation dose threshold value; and
controlling the UV radiation emitted from the at least one UV source also based on the lower UV radiation threshold value.

15. The method of claim 13, wherein controlling the UV radiation comprises at least one of: extending the emitting time and increasing the intensity of the UV radiation.

16. (canceled)

17. (canceled)

18. (canceled)

19. (canceled)

20. The method according to claim 12, further comprising:
receiving a detection of human presence form at least one human presence sensor; and
calculating the UV radiation dose based on a detected presence of at least one human.

21. (canceled)

22. A method of placing one or more UV radiation sources in an internal space, comprising:
receiving geometrical information related to an internal space comprising one or more walls;
receiving UV radiation emitting level of each one of the one or more UV sources;
determining locations of the one or more UV sources on the one or more walls such that UV radiation variations at one or more locations in the internal space will not exceed a predetermined value.

23. The method of claim 22, wherein determining the locations of the one or more UV sources comprises calculating UV radiation doses at the one or more locations in the internal space based on the UV radiation emitting level, an angular gain curve, and calibration data.

24. The method of claim 22, wherein the internal space comprises a floor and wherein the one or more locations in the internal space are located between one to two meters above the floor.

25. The method according to claim 22, wherein determining locations of one or more UV sources on the one or more walls is further such that UV radiation doses at the one or more locations in the internal space over a first pre-determined period of time will not decrease below a first UV radiation dose.

26. (canceled)

27. The method according to claim 22, wherein determining locations of one or more UV sources on the one or more walls is further such that UV radiation doses at the one or more locations in the internal space over a second predetermined period of time will not exceed a second UV radiation dose.

28. (canceled)
